(19) United States
(12) Patent Application Publication
(10) Pub. No.: US 2010/0081865 A1
(43) Pub. Date: Apr. 1, 2010

(54) Surgical method and mesh

(76) Inventor: Sam Ir Hamati, Spring Lake, MI (US)

Correspondence Address: ROBERT J. SAYFIE, P.C., 161 OTTAWA AVENUE NW, SUITE 407, GRAND RAPIDS, MI, 49503, US

(21) Appl. No.: 12/286,130

(22) Filed: Sep. 26, 2008

(51) Int. Cl. A61F 2/02; A61B 17/00

(52) U.S. Cl. 600/37; 606/151

(57) ABSTRACT

A surgical mesh, having a mesh material having a first leaf, a second leaf, and a third leaf. The first leaf having first leaf targets disposed thereon, the second leaf having second leaf targets disposed thereon, the third leaf having third leaf targets disposed thereon. The leafs may also have an orientation line longitudinally disposed thereon. A surgeon can use the targets to identify the location of where to apply sutures through the mesh material to the patient to remedy prolapse.

### GOVERNMENT RIGHTS

This invention was not made with Government support under. The Government does not have any rights in this invention.

### BACKGROUND OF THE INVENTION

Uterine prolapsed occurs when the uterus slips downward into the vagina. Slight prolapsed may be unnoticeable, but the uterus may drop down so far that its bottom portion, the cervix, is felt as a round bulge at or coming out of the vaginal opening.

In women who have had a hysterectomy, the top part of the vagina may prolapse into the lower vagina.

Surgery may be required to return the organs to their correct anatomical positions and retain them in place.

Per the National Center for Health Statistics, Hysterectomy Surveillance, U.S., 1994-1999; it is estimated that over 120,000 cases of uterine and vaginal vault prolapse are surgically treated each year in the U.S.

Sacrocolpopexy is a method of correcting prolapse by using mesh to hold the vagina in an improved anatomical position. The mesh can be applied to reposition or hold the vagina from either an open surgery incision, which may be a 15-30 cm horizontal incision, or smaller incisions, which are less invasive than the procedure using the open surgery incision. This less invasive procedure results in less blood loss and less scarring. The less invasive procedure may use instruments such as monopolar curved scissors, bipolar forceps, needle drivers, dissectors, drivers, fenestrated graspers, or cadiere forceps.

The mesh material may be sutured to the vagina, specifically the anterior vaginal wall or portion, the posterior vaginal wall or portion, and the longitudinal ligament, to reposition and hold the vagina in place. The procedure is physically challenging because of the small working environment and the limited camera viewing area of the mesh and internal organs. With small working conditions and a limited field of visibility, it is possible for the mesh to be secured to an organ in a way that applies unequal forces on the mesh and the organ, reducing the longevity of the mesh and the operation, which may result in a higher failure rate or resulting in subsequent surgeries.

As can be seen, there is a need for a surgical procedure and mesh that allows surgeons to apply sutures through targeted locations on the mesh that improve the visibility of suture location during surgery. There is also a need to enable surgeons to apply sutures through targets that are positioned to apply substantially equal forces on the mesh and organ. This improved positioning decreases creased or folded mesh. This improved positioning results in mesh that is secured to the vagina with substantially equal forces, which increases the holding and longevity of the mesh and organ.

There is also a need to improve visual orientation for fast, enhanced and secure repair. There is a need to facilitate precise, highly effective suture placement at anatomically correct pelvic organ prolapsed involving the vaginal apex. There is a need to reduce operating room (O.R.) time. There is also a need to increase safety. There is a need for a simple solution in mesh delivery and orientation. There is a need for ease in tailoring the size of the mesh done intracorporeally as necessary by using targets. There is a need to suture through marked targets to distribute equal forces. There is a need to improve mesh alignment to increase efficiency. There is also a need to maintain equal tension on the anterior and posterior vaginal wall. There is a need to decrease the failure rate. In addition, there is a need for the symmetrical placement of the sutures.

### SUMMARY OF THE INVENTION

An aspect of the present invention comprises a surgical mesh (10), comprising a first leaf (20) having a first leaf proximal end (22); a second leaf (30) having a second leaf proximal end (32); a third leaf (40) having a third leaf proximal end (42); said first leaf proximal end (22) disposed near said second leaf proximal end (32); said second leaf proximal end (32) disposed near said third leaf proximal end (42); a target (48) disposed on at least one of either said first leaf (20), said second leaf (30), or said third leaf (40).

Another aspect of the present invention is a surgical method (200) of applying a surgical mesh (10) to a patient, comprising the steps attaching a third leaf (40) to an organ (210) by using a third leaf target (70) as a reference point; attaching a second leaf (30) to an organ (220) by using a second leaf target (60) as a reference point; and attaching a first leaf (20) to an organ by using a first leaf target (50) as a reference point.

A third aspect of the present invention is a surgical mesh (10), comprising: a mesh material (90) having a first leaf (20), a second leaf (30), and a third leaf (40), said first leaf (20) having an orientation line (100) disposed on at least one of said first leaf (20), said second leaf (30), or said third leaf (40).

These and other features, aspects and advantages of the present invention will become better understood with reference to the following drawings, description and claims.

### BRIEF DESCRIPTION OF THE DRAWINGS

FIG. 1 is a pictorial of an embodiment of the surgical mesh of the present invention;

FIG. 2 is a pictorial of the leafs of an embodiment of the surgical mesh of the present invention; FIGS. 2A is the first leaf; 2B the second leaf; and 2C the third leaf;

FIG. 2 is a pictorial of the leafs of an embodiment of the surgical mesh of the present invention; FIGS. 2A is the first leaf; 2B the second leaf; and 2C the third leaf;

FIG. 3 is a pictorial of an embodiment of the mesh material and target of the present invention;

FIG. 4 is a schematic of a method of surgery of the present invention; and

FIG. 5 is a pictorial of the mesh applied on a patient.

### DETAILED DESCRIPTION OF THE INVENTION

The following detailed description is of the best currently contemplated modes of carrying out the invention. The description is not to be taken in a limiting sense, but is made merely for illustrating the general principles of the invention, since the scope of the invention is best defined by the appended claims.

### REFERENCE NUMERALS

10 surgical aid

20 first leaf

22 first leaf proximal end

24 first leaf edge

26 first leaf middle portion

30 second leaf

32 second leaf proximal end

34 second leaf edge

36 second leaf row

48 target

50 first leaf target

60 second leaf target

70 third leaf target

80 aperture or pore

90 mesh material

100 orientation line

120 first orientation line

130 second orientation line

140 third orientation line

200 surgical method

210 attaching the third leaf 40 to an organ

220 attaching the second leaf 30 to an organ

230 attaching the first leaf 20 to an organ

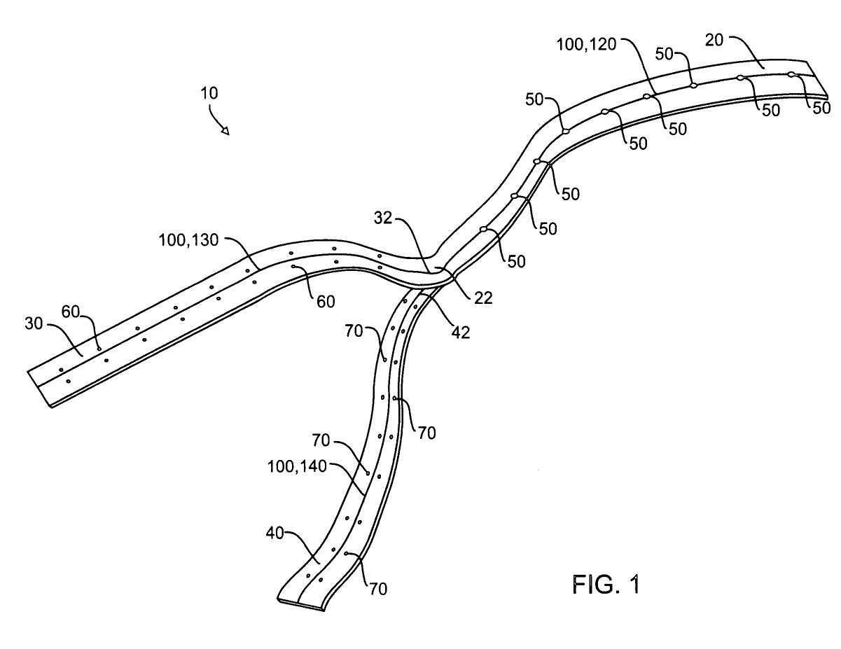

FIG. 1 illustrates one embodiment of the present invention surgical material 10, also referred to herein as surgical mesh 10. One embodiment of the surgical material 10 or surgical mesh may have a first leaf 20; the first leaf 20 may have a first leaf proximal end 22. A second leaf 30 may have a second leaf proximal end 32. A third leaf 40 may have a third leaf proximal end 42. The first leaf proximal end 22 may be connected to the second leaf proximal end 32 or the third leaf proximal end 42, or both the second leaf proximal end 32 and the third leaf proximal end 42. The third leaf proximal end 42 may be connected to either the first leaf proximal end 22, or the second leaf proximal end 32, or both.

The first leaf 20, second leaf 30, and third leaf 40 may be constructed of a mesh material 90 in one embodiment. In one embodiment, the mesh material 90 may be a large pore polypropylene mesh. One suitable type of large pore polypropylene mesh is IntePro™ of American Medical Systems, Inc. of Minnetonka, Minn. The mesh material 90 may also be a mesh elastic-elastic lightweight and large pore polypropylene, such as Optilene® of Aesculap, Inc., Center Valley, Pa.

A target 48, such as a first leaf target 50 may be disposed on the first leaf 20. The first leaf target 50 may be made of thread, or any suitable material that allows suturing therethrough. The first leaf target 50 may be made of the same mesh material 90 as the first leaf 20. The first leaf target 50 may be a different color than the remainder of the first leaf 20. In one embodiment, the first leaf target 50 may be green. The target 48, 50, 60, 70 may be visible from both sides of the respective leaf 20, 30, 40. The target 48, 50, 60, 70 may be visible on both sides of the respective leaf 20, 30, 40.

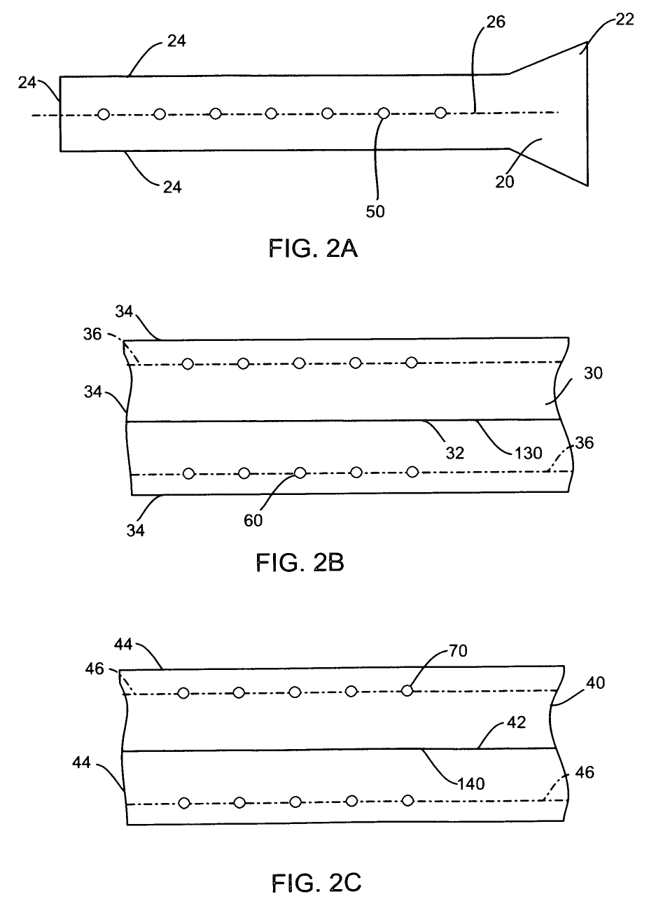

The first leaf 20 may also be referred to as the sacral wall leaf 20 because the first wall leaf 20 or first leaf 20 may be attached to the longitudinal ligament of the patient. As illustrated in FIG. 2A, the first leaf 20 may taper as the sacral leaf extends away from a sacral leaf proximal end 22.

In one embodiment the first leaf 20 may be about 4.5 inches long by about 1.5 inches wide.

In one embodiment the first leaf 20 may have green targets 50 because the word longitudinal has a “g” in it and the word “green” starts with a “g so the surgeon can possibly more easily know that the first leaf 20 is sutured to the longitudinal ligament.

Similarly, a target 48, such as a second leaf target 60 may be disposed on the second leaf 30. The second leaf target 60 may be made of thread 100 or any material that allows suturing therethrough. The second leaf target 60 may be the same mesh material 90 as the second leaf 30. The second leaf target 60 may be a different color than the remainder of the second leaf 30. The second leaf target 60 may be a different color than the first leaf target 50. In one embodiment the second leaf target 60 may be the color red. The second leaf target 60 may be red because the second leaf 30 may be sutured to the posterior wall of the vagina, which may be close to the rectum, and the word “rectum” and “red” both start with the letter “r” so that the surgeon can possibly easily identify that the leaf with the red target 60 is sutured to the wall near the rectum of the patient.

The second leaf 30 may also be referred to as the posterior wall leaf 30 because the posterior wall leaf 30 may be attached to the posterior vaginal wall or the rectum of the patient.

In one embodiment the second leaf 30 may be about 3 inches long by about 1.5 inches wide.

A target 48, such as a third leaf target 70, may be disposed on the third leaf 40, also referred to as the anterior wall leaf 40. The third leaf target 60 may be made of thread 100. The third leaf target 70 may be the same mesh material 90 as the third leaf 40. The third leaf target 70 may be a different color than the remainder of the third leaf 40. The third leaf target 70 may be a different color than the first leaf target 50. The third leaf target may be a different color than the second leaf target 60. In one embodiment the third leaf target 70 may be blue. The third leaf target 70 may be blue because the third leaf 40, also referred to herein anterior wall leaf 40 is close to the bladder; and the word “bladder” and “blue” both start with the letter “b.”

The third leaf 40 may also be referred to as the anterior wall leaf 40 because the anterior wall leaf 40 may be attached to the anterior vaginal wall or the bladder of the patient.

In one embodiment the third leaf 40 may be about 3 inches long by about 1.5 inches wide.

As illustrated in FIG. 2A the first leaf 20 may have a single row of first leaf targets 50 disposed along a first leaf middle portion 26 of the first leaf 20.

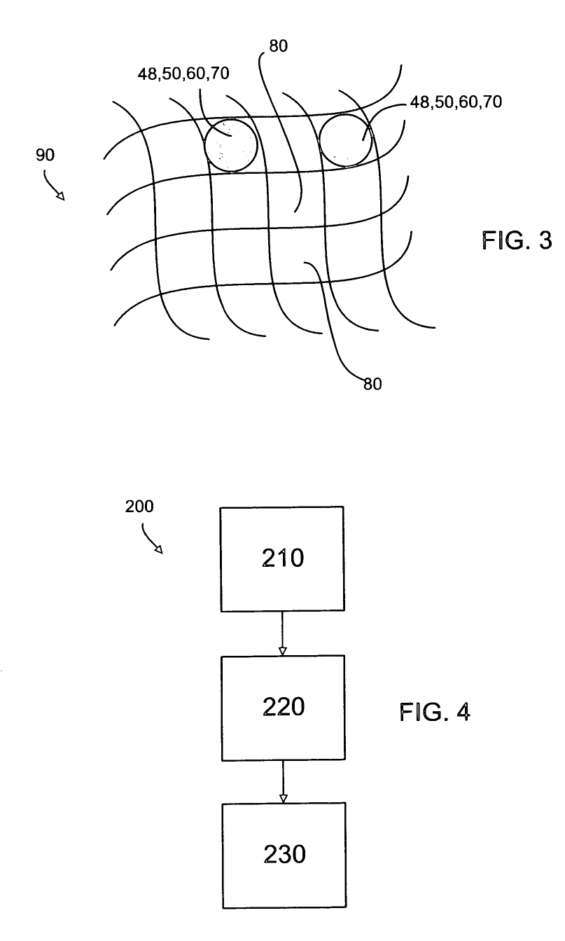

As illustrated in FIG. 2B the second leaf 30 may have two second leaf rows 36 of second leaf targets 60 disposed thereon. Each second leaf row 36 may be disposed about 1.5 cm from the second leaf edge 34. The second leaf edge 34 may be disposed about the perimeter of the second leaf 30. Alternatively each second leaf row 36 may be disposed so that there are at least two apertures 80 or pores 80 from the third second leaf edge 34 to the target 60, as illustrated in FIG. 3.

As illustrated in FIG. 2C the third leaf 40 may have two third leaf rows 46 of third leaf targets 70 disposed thereon. Each third leaf row 46 may be disposed about 1.5 com from the third leaf edge 44. The third leaf edge 44 may be disposed about the perimeter of the third leaf 40. Alternatively each third leaf row 46 may be disposed so that there are at least two apertures 80 or pores 80 from the third leaf edge 44 to the target 70, as illustrated in FIG. 3.

As illustrated in FIG. 3, in one embodiment the respective targets 50, 60, 70 are disposed with at least 2 apertures or pores 80 inwardly from the respective edge 24, 34, 44, such as the first leaf edge 24, the second leaf edge 34, or the third leaf edge 44. This may reduce tears of the mesh material 90 and provide for a more stable attachment of the mesh material 90 to the organ of the patient.

In another embodiment the targets 50, 60, 70 may be disposed inwardly from the respective edges 24, 34, 44 by a distance of about 1.5 cm. In other words, there may be a distance of about 1.5 cm of mesh material 90 from an edge of the target 50, 60, 70 to the respective edge 24, 34, 44 of the mesh material 90. The present invention may be used with mesh material 90 having different sized apertures 80; or with differently sized mesh material 90.

In one embodiment the second leaf 30 or posterior wall leaf 30 may be longer than the third leaf 40 or the anterior wall leaf 40.

As illustrated in FIGS. 1, 2B, and 2C, an orientation line 100 may be disposed longitudinally along at least one of first leaf 20, second leaf 30, or third leaf 40. The first leaf 20 may have a first orientation line 120 disposed longitudinally thereon. The second leaf 30 may have a second orientation line 130 disposed longitudinally thereon. The third leaf 40 may have a third orientation line 140 disposed longitudinally thereon. The orientation line 100, 120, 130, 140 may be visible from either side of the respective leaf 20, 30, 40. The orientation line 100, 120, 130, 140 may be visible on both sides of the respective leaf 20, 30, 40.

In one embodiment the first orientation line 120 is the same color as the first leaf target 50. In one embodiment the second orientation line 130 is the same color as the second leaf target 60. In one embodiment the third orientation line 140 is the same color as the third leaf target 70. The first orientation line 120 may be disposed along the middle portion of the first leaf 20 and may bisect the leaf forming two substantially equal halves on each side of the first orientation line 120. The second orientation line 130 may be disposed along the middle portion of the second leaf 30 and may bisect the leaf forming two substantially equal halves on each side of the second orientation line 130. The third orientation line 140 may be disposed along the middle portion of the third leaf 40 and may bisect the leaf forming two substantially equal halves on each side of the third orientation line 140.

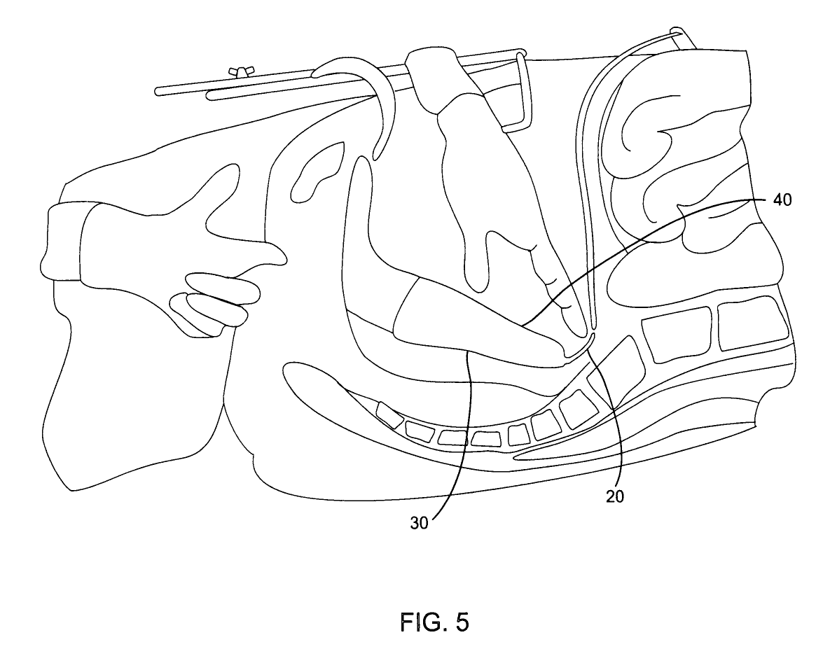

FIG. 4 illustrates one embodiment of performing the surgical method 200 with the surgical mesh 10 of the present invention. One embodiment may include the following steps:

210 attaching the third leaf 40 to an organ by using the third leaf target 70 as a reference point;

220 attaching the second leaf 30 to an organ by using the second leaf target 60 as a reference point;

230 attaching the first leaf 20 to an organ by using the first leaf target 50 as a reference point.

In one embodiment the targets 70, 60, 50 may be used as reference points by placing sutures through the respective reference point to the organ of the patient.

In one embodiment the step 210 may include the third leaf 40 attached to the anterior vaginal wall, in step 220 the second leaf 30 may be attached to the posterior vaginal wall, and in step 230 the first leaf 20 may be attached to the sacrum.

The attaching of the three leafs 40, 30, 20 may be accomplished by suturing thread through the targets 70, 60, 50 and through the respective organ of the patient so that the surgical mesh 10 holds and maintains the organs in an acceptable anatomical position.

FIG. 5 illustrates the mesh 10 of the present invention as applied on a patient.

It should be understood, of course, that the foregoing relates to exemplary embodiments of the invention and that modifications may be made without departing from the spirit and scope of the invention as set forth in the following claims.

I claim:

1. A surgical mesh (10), comprising:
a first leaf (20) having a first leaf proximal end (22);
a second leaf (30) having a second leaf proximal end (32);
a third leaf (40) having a third leaf proximal end (42);
said first leaf proximal end (22) disposed near said second leaf proximal end (32);
said second leaf proximal end (32) disposed near said third leaf proximal end (42);
a target (48) disposed on at least one of either said first leaf (20), said second leaf (30), or said third leaf (40).

2. The surgical mesh (10) of claim 1, further comprising:
a first leaf target (50) disposed on said first leaf (20);
a second leaf target (60) disposed on said second leaf (30); and
a third leaf target (70) disposed on said third leaf (40).

3. The surgical mesh (10) of claim 1, further comprising:
a plurality of said targets (48) disposed in a linear fashion to form a row near a first leaf middle portion (26).

4. The surgical mesh (10) of claim 1, further comprising:
a first leaf target (50) disposed on said first leaf (20) at least about 1.5 cm inwardly from a first leaf edge (24).

5. The surgical mesh (10) of claim 1, further comprising:
a second leaf target (60) disposed on said second leaf (30) at least about 1.5 cm inwardly from a second leaf edge (34).

6. The surgical mesh (10) of claim 1, further comprising:
a third leaf target (70) disposed on said third leaf (40) at least about 1.5 cm inwardly from a third leaf edge (44).

7. The surgical mesh (10) of claim 1, further comprising:
a first leaf target (50) disposed on said first leaf (20) whereby at least two apertures (80) separate said first leaf target (50) from a first leaf edge (24).

8. The surgical mesh (10) of claim 1, further comprising:
a second leaf target (60) disposed on said second leaf (30) whereby at least two pores (80) separate said second leaf target (60) from a second leaf edge (34).

9. The surgical mesh (10) of claim 1, further comprising:
a third leaf target (70) disposed on said third leaf (40) whereby at least two apertures (80) separate said third leaf target (70) from a third leaf edge (44).

10. The surgical mesh (10) of claim 1, further comprising:
targets (48) disposed on a first leaf middle portion (26), targets (48) disposed on two second leaf rows (36), and targets (48) disposed on two third leaf rows (46).

11. The surgical mesh (10) of claim 1, further comprising:
an orientation line (100) disposed on at least one of said first leaf (20), said second leaf (30), or said third leaf (40), and said orientation line (100) is visible from both sides of at least one of said first leaf (20), said second leaf (30), or said third leaf (40).

12. The surgical mesh (10) of claim 1, further comprising:
a first orientation line (120) disposed longitudinally along said first leaf (20);
a second orientation line (130) disposed longitudinally along said second leaf (30); and
a third orientation line (140) disposed longitudinally along said third leaf (40).

13. The surgical mesh (10) of claim 1, wherein said target (48) is visible from both sides of said first leaf (20), said second leaf (30), or said third leaf (40).

14. A surgical method (200) of applying a surgical mesh (10) to a patient, comprising the steps:
attaching a third leaf (40) to an organ (210) by using a third leaf target (70) as a reference point;
attaching a second leaf (30) to an organ (220) by using a second leaf target (60) as a reference point; and
attaching a first leaf (20) to an organ by using a first leaf target (50) as a reference point.

15. The surgical method of claim 14, wherein said third leaf (40) is attached to the anterior vaginal wall.

16. The surgical method of claim 14, wherein said second leaf (30) is attached to the posterior vaginal wall.

17. The surgical method of claim 14, wherein said first leaf (20) is attached to the longitudinal ligament of the patient.

18. A surgical mesh (10), comprising:
a mesh material (90) having a first leaf (20), a second leaf (30), and a third leaf (40), said first leaf (20) having an orientation line (100) disposed on at least one of said first leaf (20), said second leaf (30), or said third leaf (40).

19. The surgical mesh (10) of claim 18, further comprising:
a plurality of said targets (48) disposed on at least one of said first leaf (20), said second leaf (30), or said third leaf (40).

20. The surgical mesh (10) of claim 19, whereby said target (48) and said orientation line (100) are both visible from both sides of the respective said first leaf (20), said second leaf (30) and said third leaf (40).
